(19) United States
(12) Patent Application Publication
(10) Pub. No.: US 2009/0048623 A1
(43) Pub. Date: Feb. 19, 2009

(54) Medical Implement Particularly Useful in Arthroscopic Surgical Procedures

(75) Inventors: Laurent Lafosse, Annecy-Le-Vieux (FR); Ran Oren, Doar-Na Oshrat (IL); Dan Moor, Doar Na Oshrat (IL)

Correspondence Address: Martin D. Moynihan;PRTSI, P.O. Box 16446, Arlington, VA, 22215, US

(73) Assignee: T.A.G. Medical Products a Limited Partnership, Doar-Na Oshrat (IL)

(21) Appl. No.: 12/084,699

(22) PCT Filed: Nov. 9, 2006
(86) PCT No.: PCT/IL2006/001290

§ 371 Date: May 8, 2008

Related U.S. Application Data
(60) Provisional application No. 60/735,205, filed on Nov. 10, 2005.

(51) Int. Cl. A61B 17/00

(52) U.S. Cl. 606/192; 606/190

(57) ABSTRACT

A medical implement for separating tissue at a surgical site includes an elongated rod carrying an inflatable balloon of triangular configuration having first and second apices defining a base for engagement with one tissue layer, and a third apex engageable with a second tissue layer effective, upon the inflation of the balloon, to separate the tissue layers. Also described are the use of the medical implement in the performance of arthroscopic surgical procedures in the shoulder of a patient for creating a free space between the deltoid muscle or the glenoid socket and the humerus bone.

### FIELD AND BACKGROUND OF THE INVENTION

The present application relates to a medical implement particularly useful in arthroscopic surgical procedures, and also to certain arthroscopic procedures in which such medical implement is used. The novel medical implement is particularly useful in arthroscopic surgical procedures performed in the shoulder of a patient, and is therefore described below with respect to such procedures.

In all surgical procedures it is necessary to create a space for manipulating the instruments needed to perform the procedure. Overlying and adjacent soft tissue often obstruct access to the surgical site. Various means are in current use for freeing space around the site. For example, in laparoscopy when operating on joints, gas or other fluid is introduced below soft tissue to distend the area; in fully open procedures, retractors are used. However, in all these methods, space is created over the entire surface of the soft tissue and not where it is most needed.

It is known to use inflatable balloons for creating free space, but the known devices are not particularly suitable for arthroscopic surgical procedures particularly those involved in the shoulder of a patient. For example, U.S. Pat. No. 5,331,975 discloses such a medical implement for use in performing carpal tunnel surgery; U.S. Pat. No. 5,514,153 discloses a similar medical instrument; U.S. Pat. No. 5,720,762 discloses a medical instrument for surgical flap dissection; and U.S. Pat. No. 5,318,586 discloses a medical instrument including an expandable balloon for holding back the edges of a wound during a surgical procedure.

### OBJECTS AND BRIEF SUMMARY OF THE PRESENT INVENTION

An object of the present invention is to provide a medical implement particularly useful in the performance of an arthroscopic surgical procedure in the shoulder of a patient.

According to one aspect of the present invention, there is provided a medical implement for separating tissue layers at a surgical site, comprising an elongated rod having at least one lumen therethrough, a proximal end for grasping and manipulating by a user, and a distal end insertable via an incision into a surgical site; the lumen being open at the proximal end of the elongated rod for introducing a fluid, the lumen being closed at the distal end of the elongated rod; and an inflatable balloon carried by the outer surface of the elongated rod adjacent to the distal end; the interior of the balloon communicating with the lumen such that the portion of the elongated rod carrying the balloon may be inserted, by pressing the tapered distal end through the incision, into a surgical site, with the balloon in its deflated condition and located between the tissue layers to be separated, whereupon the balloon may be inflated by a fluid introduced into the lumen via the proximal end of the elongated rod to thereby separate the tissue layers; the balloon being of triangular configuration having a center coaxial with the axis of the rod, first and second apices defining a base for engagement with one tissue layer, and a third apex engageable with a second tissue layer effective, upon the inflation of the balloon, to separate the tissue layers.

According to another aspect of the present invention, there is provided a medical implement for separating tissue layers at a surgical site, comprising an elongated rod having at least one lumen therethrough, a proximal end for grasping and manipulating by a user, and a distal end insertable via an incision into a surgical site; the lumen being open at the proximal end of the elongated rod for introducing a fluid, the lumen being closed at the distal end of the elongated rod; an inflatable balloon carried by the outer surface of the elongated rod adjacent to the closed distal end; the interior of the balloon communicating with the lumen such that the portion of the elongated rod carrying the balloon may be inserted, by pressing the tapered distal end through the incision, into a surgical site, with the balloon in its deflated condition and located between the tissue layers to be separated, whereupon the balloon may be inflated by a fluid introduced into the lumen via the proximal end of the elongated rod to thereby separate the tissue layers; the balloon being of triangular configuration having a center coaxial with the axis of the rod, first and second apices defining a base for engagement with one tissue layer, and a third apex engageable with a second tissue layer effective, upon the inflation of the balloon, to separate the tissue layers; and a valve carried by the proximal end of the elongated rod, the valve being selectively opened to permit inflation of the balloon or closed to maintain the balloon inflated.

According to a further aspect of the present invention, thus provided a medical implement for separating tissue layers at a surgical site, comprising an elongated rod having at least one lumen therethrough, a proximal end for grasping and manipulating by a user, and a distal end insertable via an incision into a surgical site; the lumen being open at the proximal end of the elongated rod for introducing a fluid, the lumen being closed at the distal end of the elongated rod; the distal end of the elongated rod having a tapered outer surface to facilitate inserting the elongated rod via said incision into the surgical site; and an inflatable balloon carried by the outer surface of the elongated rod adjacent to the distal end; the interior of the balloon communicating with the lumen of the elongated rod such that the portion of the elongated rod carrying the balloon may be inserted, by passing said tapered distal end through the incision, into a surgical site, with the balloon in its deflated condition and located between the tissue layers to be separated, whereupon the balloon may be inflated by a fluid introduced into the lumen via the proximal end of the elongated rod to thereby separate the tissue layers.

As will be described more particularly below, such a medical implement is particularly useful in the performance of an arthroscopic surgical procedure in the shoulder of a patient. Two such surgical procedures are described below. In one, the medical implement is used for creating a free space between the deltoid muscle and the humerus bone; whereas in the other described procedure, it is used for creating a free space between the glenoid socket and the humerus bone.

Further features and advantages of the invention will be apparent from the description below.

### BRIEF DESCRIPTION OF THE DRAWINGS

The invention is herein described, by way of example only, with reference to the accompanying drawings, wherein:

FIG. 1 schematically illustrates one form of medical implement constructed in accordance with present invention particularly for use during an arthroscopic surgical procedure on a person's shoulder;

FIG. 2 is an enlarged fragmentary view more particularly illustrating the balloon-carrying rod of the medical implement of FIG. 1;

FIG. 3 illustrates a balloon-carrying rod as in FIG. 2 but constructed so as to be deformable;

FIG. 4 illustrates a rigid core particularly useful with the flexible balloon-carrying rod as shown in FIG. 3 to facilitate its insertion into the surgical site;

FIGS. 5a and 5b are side and end views, respectively, illustrating a rod carrying a plural-compartment balloon of triangular configuration;

FIGS. 6a-8a and 6b-8b, respectively, illustrate other balloons of various configurations in their deflated and inflated conditions, respectively;

FIG. 9 is a schematic cross-section of a shoulder joint;

FIG. 10 illustrates the use of a medical implement constructed in accordance with the present invention for creating a free space between the deltoid muscle and the humerus bone during an arthroscopic surgical procedure on the shoulder of a patient; and

FIG. 11 illustrates the use of the medical implement in a similar surgical procedure for creating free space between the glenoid socket and the humerus bone.

It is to be understood that the foregoing drawings, and the description below, are provided primarily for purposes of facilitating understanding the conceptual aspects of the invention and possible embodiments thereof, including what are presently considered to be preferred embodiments. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF PREFERRED EMBODIMENTS

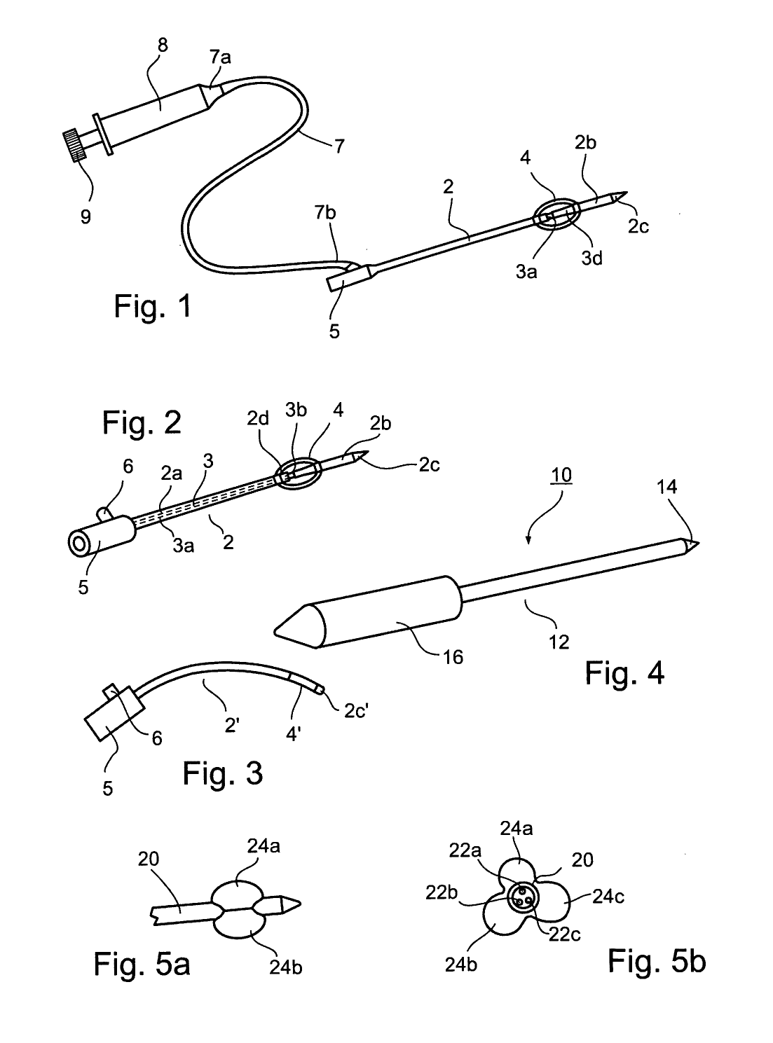

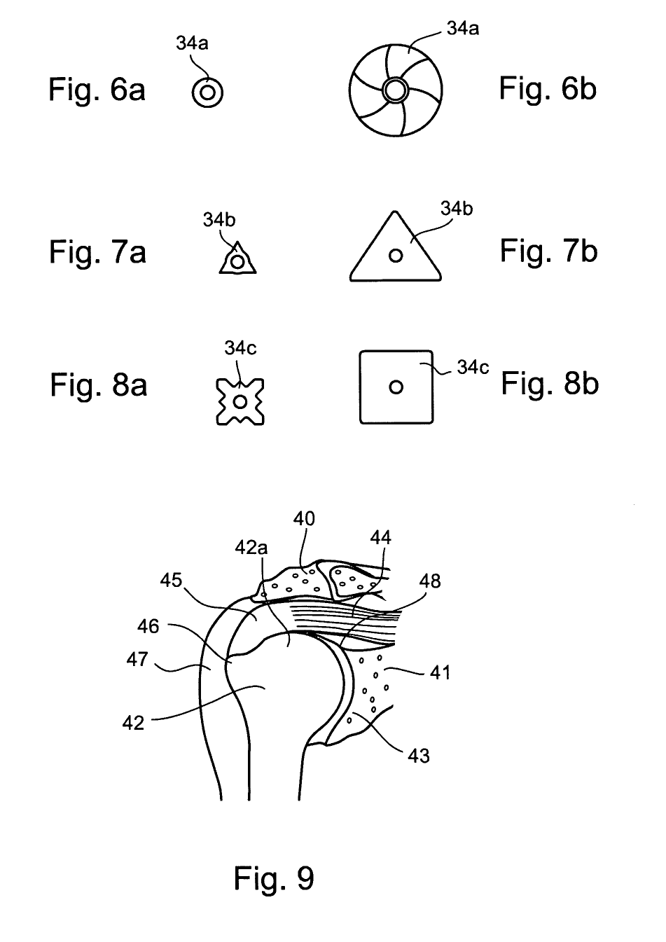

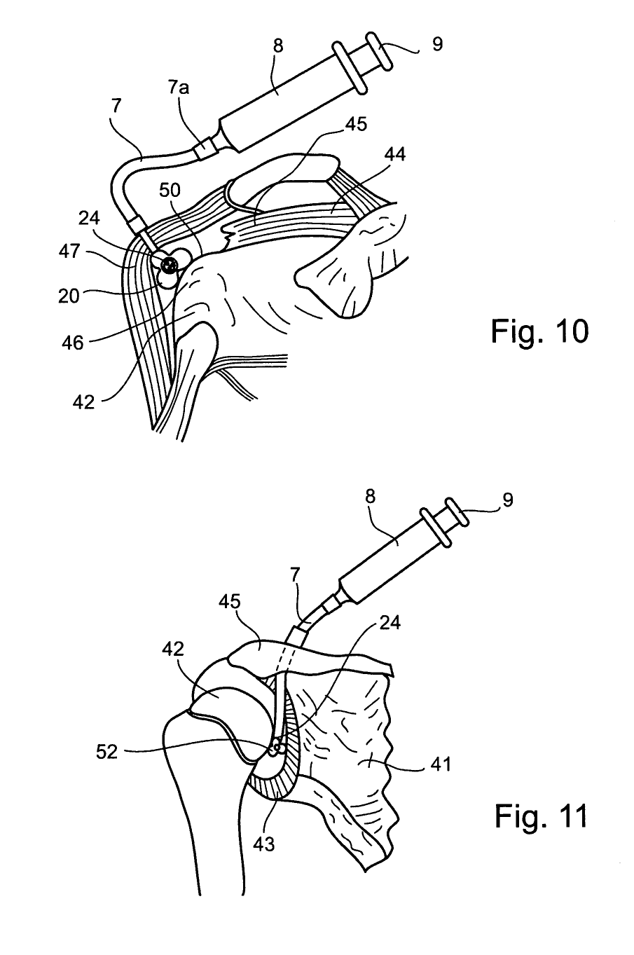

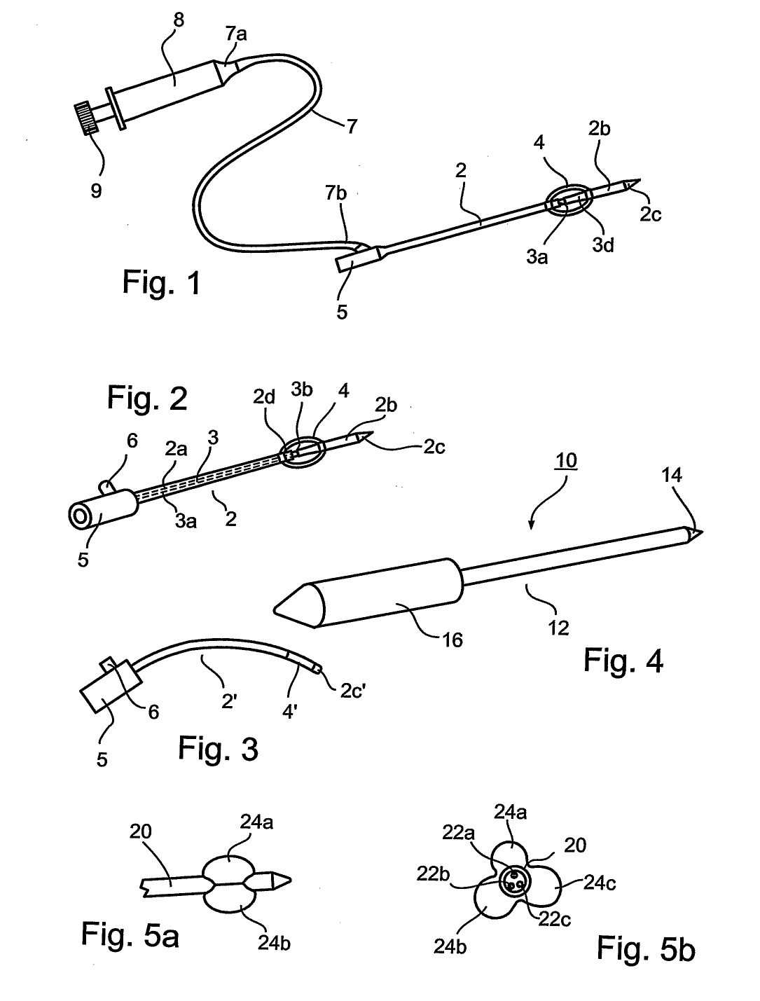

As indicated below, the invention provides a medical implement particularly useful in arthroscopic surgery on a person's shoulder for creating a free working space between various types of tissue. FIGS. 1 and 2 schematically illustrate one form of medical implement constructed in accordance with the present invention for this purpose.

The medical implement illustrated in FIG. 1, as more particularly shown in FIG. 2, includes an elongated rod 2 having at least one lumen 3 (FIG. 2) therethrough extending from the proximal end 2a of the elongated rod but terminating short of its distal end 2b. The proximal end 2a of elongated rod 2 includes a handle 4 graspable by the user, e.g. a surgeon, for manipulating the rod; whereas the distal end 2b is formed with a somewhat pointed tip 2c having a tapered outer surface to facilitate inserting the elongated rod via an incision into the surgical site.

An inflatable sleeve or balloon 5 is carried by the outer surface of elongated rod 2 adjacent to its distal end 2b. The proximal end 3a of lumen 3 is open for introducing a fluid therethrough, whereas the distal end 3b of the lumen is closed but communicates via an opening 3c with the interior of balloon 5 so as to enable inflation of the balloon by the fluid introduced via the lumen proximal end 3a.

Handle 4 at the proximal end 2a of elongated rod 2 carries a valve, schematically shown at 6, having a manual operator 6a enabling the valve to be manually opened or closed. As will be described below, valve 6 would be opened to enable inflation of balloon 5, and closed to maintain the balloon in its inflated condition.

As shown in FIG. 1, the illustrated medical implement further includes a flexible tube 7 connected at one end 7a to a syringe 8, and at the opposite end 7b to valve 6. Syringe 8 includes a plunger 9, which may be manually depressed in order to force the contents of the syringe, e.g. saline water or a gas, via tube 7 and lumen 3 of elongated rod 2, to inflate balloon 5.

The manner of using the medical implement illustrated in FIGS. 1 and 2 will be apparent from the above description. Thus, handle 4 at the proximal end 2a of elongated rod 2 is grasped by the user, e.g. the surgeon, and is manipulated to pass the pointed distal tip 2c of the rod through the incision into the surgical site, and to locate balloon 5 between the tissue to be separated. When the balloon 5 is so located, plunger 9 of syringe 8 is pressed inwardly to force the fluid therein through tube 7 and lumen 3, to inflate balloon 5, and thereby to create a working space between the two tissues. At that time, valve operator 6a may be pressed to close valve 6, and thereby to maintain balloon 5 inflated during the surgical procedure involved.

After the surgical procedure has been completed, valve 6 is opened, and plunger 9 is pulled outwardly, to thereby deflate balloon 5, whereupon the elongated rod 2, with the deflated balloon 5, may be removed from the surgical site.

Elongated rod 2, and balloon 5 carried at its distal end, are made of any suitable biocompatible material. Preferably, elongated rod 2 is of reduced outer diameter at the portion thereof occupied by balloon 5, as shown at 2d in FIG. 2, to reduce the overall diameter of the distal end of the elongated rod, and thereby to facilitate its introduction, with the deflated balloon, into the incision to the surgical site. Preferably, balloon 5 is made of a suitable elastomer or plastomer which provides adhesion with the outer surface of the elongated rod. Elongated rod 2 is preferably made of a rigid material to facilitate its insertion and manipulation in the surgical site.

FIG. 3 illustrates a modification wherein the elongated rod, therein designated 2′, is deformable to facilitate manipulation of its balloon 5′ at the distal end of the rod to the surgical site. FIG. 4 illustrates a rigid core 10 which may be used when a deformable elongated rod, such as shown at 2′ in FIG. 3, is used. Rigid core 10 includes a main section 12 dimensioned to be inserted within lumen 3 of the elongated rod 2′, and is formed with a pointed distal tip 14 to facilitate the insertion of the core into the elongated rod and its manipulation with the deflated balloon to the surgical site. The proximal 16 end of core 10 has an outer triangular configuration to enable better grasping and manipulating it into the elongated rod. After rod 2 has been inserted into the surgical site, with the balloon 5 properly located between the two layers of tissue to be separated, the rigid core 10 is withdrawn from the rod, and lumen 3, through which the rod was inserted, is now used for inflating balloon 5.

Valve 6 is constructed so that it automatically opens upon the insertion of core 10 into lumen 3, and automatically closes upon the removal of the core from the lumen. The valve then remains closed until depressed by its operator 6a to open the valve.

For inserting the above-described implement into the surgical site, a small incision is first made through the skin; and a trocar is then used to open a channel through the muscles overlying the joint to allow for access. The implement is introduced through this channel to locate its balloon, when deflated, at the proper location between the tissue layers to be separated. Elongated rod 2 normally should be somewhat deformable or flexible to enable maneuvering it to the desired location, in which case the rigid core 10 of FIG. 4 would be used for positioning the elongated rod, together with its deflated balloon, and then removed before inflation of the balloon.

FIGS. 5a and 5b illustrate a preferred construction of the medical implement, in that, instead of there being a balloon having a single compartment at the distal end of the elongated rod 20, the balloon 24 is divided into three separate compartments 24a, 24b, 24c, each communicating with a separate lumen 22a, 22b, 22c through the elongated rod 20 to enable selective inflation of each compartment. For example, according to the conditions at the surgical site, it may be desirable to inflate only one compartment of the balloon, or only two compartments, or to inflate the compartments to a different volume, in order to create the desired free space according to the conditions at the surgical site.

It will thus be seen that the three-compartment balloon illustrated in FIGS. 5a and 5b define a balloon of a triangular configuration, having a center coaxial with the axis of the elongated rod 20, first and second apices defined by the outer surfaces of the compartment 24a and 24b which may serve as a base for engagement with one tissue layer, and a third apex defined by the outer surface of department 24c engageable with the second tissue layer effective, upon the inflation of the balloon, to separate the two tissue layers from each other, and thereby to facilitate the surgical procedure required.

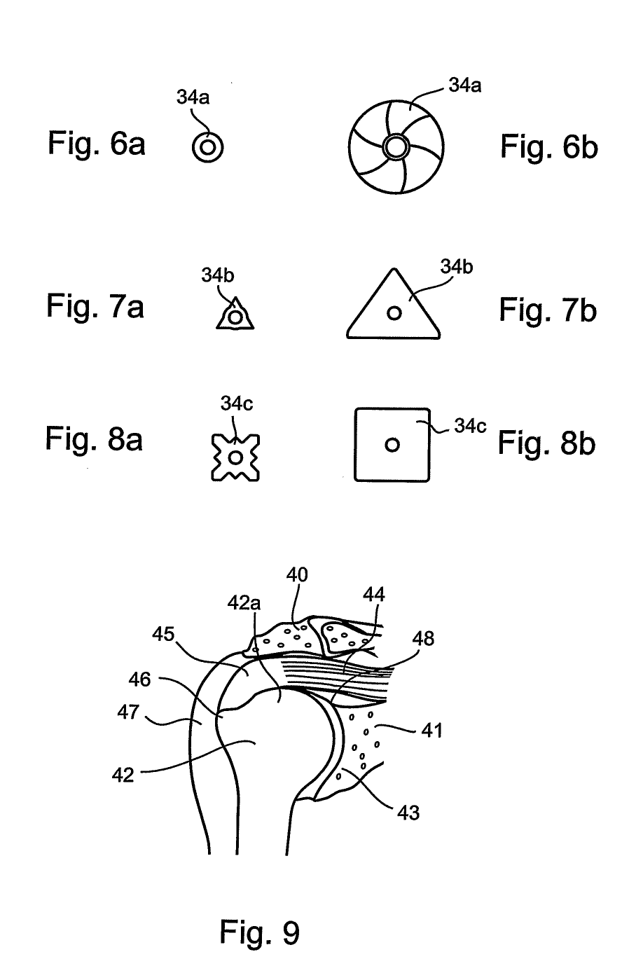

FIGS. 6a-8a and 6b-8b illustrate the deflated and inflated conditions, respectively, of other different configurations of balloons which may be provided at the distal end of the elongated rod. Thus, FIGS. 6a and 6b illustrate a single-compartment balloon 34a of circular configuration; FIGS. 7a and 7b illustrate a single-compartment balloon 34b of triangular configuration; and FIGS. 8c and 8b illustrate a single-compartment balloon 34c of polygonal, particularly rectangular, annular configuration. It will be appreciated still other configuration could be used according to the particular conditions at the surgical site. When one of the foregoing single-compartment configurations is used, the triangular configuration of FIGS. 7a and 7b is preferred since, like the triangular configuration of FIGS. 5a and 5b, the inflated balloon defines two apices serving as a base for engagement with one tissue layer, and a third apex engageable with the second tissue layer and effective, upon the inflation of the balloon, to separate the two tissue layers.

As indicated earlier, the medical implement described above is particularly useful for creating free spaces during arthroscopic surgery in the shoulder of a patient. FIG. 9 schematically illustrates a coronal section of the human shoulder. The bones visible are the acromium 40 and the glenoid 41, both of which are parts of the shoulder-blade, and the humerus 42. The glenoid socket 43 and the head of the humerus 42 form the ball-and-socket joint. A group of muscles, known as the rotator cuff, surround the joint; the supraspinatus, shown at 44, is one of these muscles. The supraspinatus is attached to the humerus by its tendon 41 laterally, extending to the greater tubercle 46 of the humerus. The deltoid muscle 47 is attached to the acromium 40 and the stem of the humerus. A soft fibrous tissue, the labrum 48, surrounds the glenoid socket.

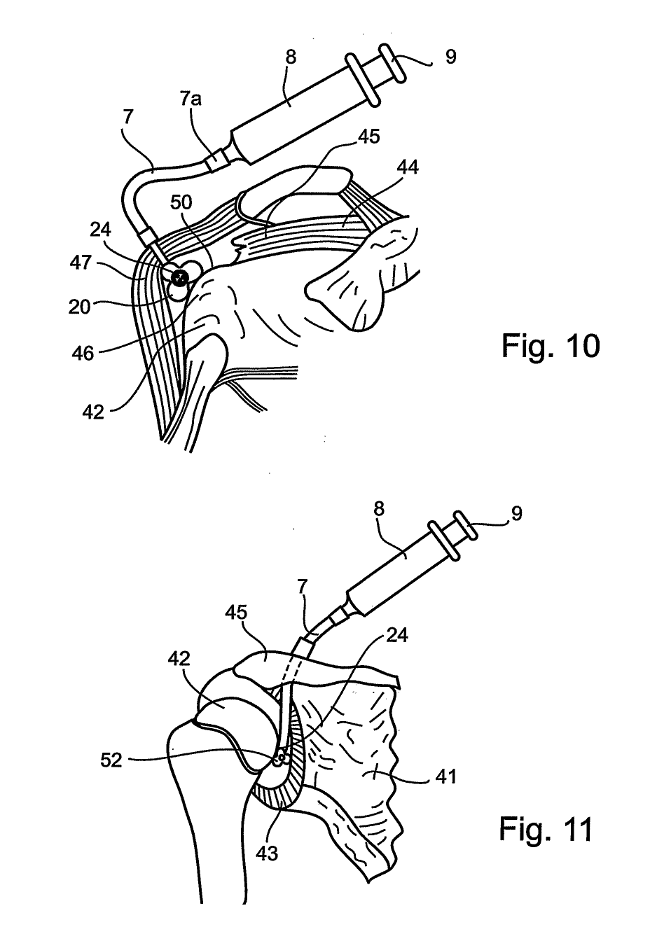

The use of the invention in certain arthroscopic procedures, by way of example, is clearly described below with reference to FIG. 10 and FIG. 11.

In FIG. 10, the deltoid muscle 47 is seen overlying the supraspinatus tendon 45 of the rotator cuff and the greater tubercle 46 of the humerus. The supraspinatus 44 is normally laterally connected to the head of the humerus bone 42 as shown in FIG. 9. FIG. 10 illustrates a pathological situation, wherein the supraspinatus 44 is detached from the humerus 42. In such cases. it becomes necessary to reattach the muscle to the bone surgically by applying a number of anchors to the bone to cover the area needed for reattachment.

Sutures connected to the anchors are passed through the supraspinatus 44 and serve to proximate the tissue to the bone to restore its anatomical position. The fixation to the bone is preferably performed arthroscopically. Drilling and placing the anchors can be easily performed through the overlying tissue; the suturing, however, needs space over the supraspinatus 44. Introducing fluid between the muscles does not lift the deltoid 47 sufficiently to allow easy working in the lateral section, as needed for an anchor at position 50.

FIG. 10 illustrates the use of the described implement in this situation. By using the implement shown in FIG. 1, using the rod 20 and three-compartment balloon 24 of FIGS. 5a and 5b, the surgeon introduces rod 20 transversally into the joint and manipulates the inflatable balloon 24 to be in position under the deltoid 47 as shown. Connecting the syringe to end 7a of tube 7, the surgeon inflates balloon 24 as needed for easy working. Valve 6 keeps the balloon inflated while the syringe is removed. Reconnecting the syringe opens the valve to permit deflating.

The use of the described implement in a different arthroscopic procedure in the shoulder is illustrated in FIG. 11.

As shown in FIG. 9, the humeral head 42a normally rests in the shallow glenoid socket 43. Because the head of the upper arm bone is usually much larger than the socket, the labrum 48 surrounds the socket to help stabilize the joint. The labrum deepens the socket by up to 50 percent so that the head of the upper arm bone fits better. The muscles of the rotator cuff keep the cartilages covering both the glenoid 41 and the humeral head 42a in close contact. The labrum 48 also serves as an attachment site for several ligaments. In arthroscopic procedures involving the glenoid rim 41 and the labrum 48, the proximity of the large humeral head poses a difficulty.

Positioning balloon 24 between the bones, in position 52 in FIG. 11, and inflating it, creates a space for easier working around the glenoid 41.

While the invention has been described with respect to several preferred embodiments, it will be appreciated that these are set forth merely for purposes of example, and that many other variations, modifications and applications of the invention may be made.

What is claimed is:

1. A medical implement for separating tissue layers at a surgical site, comprising:
an elongated rod having at least one lumen therethrough, a proximal end for grasping and manipulating by a user, and a distal end insertable via an incision into a surgical site;
the lumen being open at the proximal end of the elongated rod for introducing a fluid, the lumen being closed at the distal end of the elongated rod;
and an inflatable balloon carried by the outer surface of the elongated rod adjacent to said distal end;
the interior of said balloon communicating with said lumen such that the portion of the elongated rod carrying said balloon may be inserted, by pressing said tapered distal end through said incision, into a surgical site, with the balloon in its deflated condition and located between the tissue layers to be separated, whereupon the balloon may be inflated by a fluid introduced into said lumen via the proximal end of the elongated rod to thereby separate said tissue layers;
said balloon being of triangular configuration having a center coaxial with the axis of said rod, first and second apices defining a base for engagement with one tissue layer, and a third apex engageable with a second tissue layer effective, upon the inflation of said balloon, to separate said tissue layers.

2. The medical implement according to claim 1, wherein said balloon has a single compartment communicating with said lumen.

3. The medical implement according to claim 1, wherein said balloon has three compartments, one including each of said apices, and said rod has three lumens each communicating with one of said compartments.

4. The medical implement according to claim 1, wherein said proximal end of the elongated rod includes a valve which may be selectively opened to permit inflation of said balloon, or closed to maintain said balloon inflated.

5. The medical implement according to claim 4, wherein said medical implement further includes a syringe connected to said proximal end of the elongated rod for inflating said balloon when said valve is open.

6. The medical implement according to claim 5, wherein said syringe is connected to said elongated rod by a flexible tube attachable at its opposite ends to connectors carried by said syringe and proximal end of the elongated rod, respectively.

7. The medical implement according to claim 1, wherein the portion of said elongated rod carrying said balloon is of reduced diameter to facilitate inserting the elongated rod via said incision into the surgical site.

8. The medical implement according to claim 1, wherein said distal end of the elongated rod has a tapered outer surface to facilitate inserting it via said incision into the surgical site.

9. The medical implement according to claim 1, wherein said rod is deformable, and said medical implement further includes a rigid core insertable into said lumen of the rod for stiffening the rod when introduced into the surgical site, and removable therefrom when the rod in proper position and before the balloon is inflated.

10. A medical implement for separating tissue layers at a surgical site, comprising:
an elongated rod having at least one lumen therethrough, a proximal end for grasping and manipulating by a user, and a distal end insertable via an incision into a surgical site;
the lumen being open at the proximal end of the elongated rod for introducing a fluid, the lumen being closed at the distal end of the elongated rod;
an inflatable balloon carried by the outer surface of the elongated rod adjacent to said closed distal end; the interior of said balloon communicating with said lumen such that the portion of the elongated rod carrying said balloon may be inserted, by pressing said tapered distal end through said incision, into a surgical site, with the balloon in its deflated condition and located between the tissue layers to be separated, whereupon the balloon may be inflated by a fluid introduced into said lumen via the proximal end of the elongated rod to thereby separate said tissue layers;
said balloon being of triangular configuration having a center coaxial with the axis of said rod, first and second apices defining a base for engagement with one tissue layer, and a third apex engageable with a second tissue layer effective, upon the inflation of said balloon, to separate said tissue layers;
and a valve carried by said proximal end of the elongated rod, said valve being selectively opened to permit inflation of the balloon or closed to maintain said balloon inflated.

11. The medical implement according to claim 10, wherein said medical implement further includes a syringe connectable to said proximal end of the elongated rod for inflating said balloon.

12. The medical implement according to claim 10, wherein said balloon is of triangular configuration having a center coaxial with the axis of said rod, first and second apices defining a base for engagement with one tissue layer, and a third apex engageable with a second tissue layer effective, upon the inflation of said balloon, to separate said tissue layers.

13. The medical implement according to claim 12, wherein said balloon has a single compartment communicating with said lumen.

14. The medical implement according to claim 12, wherein said balloon has three compartments, one including each of said apices, and said rod has three lumens each communicating with one of said compartments.

15. The medical implement according to claim 10, wherein said distal end of the elongated rod has a tapered outer surface to facilitate inserting it via said incision into the surgical site.

16. A medical implement for separating tissue layers at a surgical site, comprising:
an elongated rod having at least one lumen therethrough, a proximal end for grasping and manipulating by a user, and a distal end insertable via an incision into a surgical site;
the lumen being open at the proximal end of the elongated rod for introducing a fluid, the lumen being closed at the distal end of the elongated rod;
said distal end of the elongated rod having a tapered outer surface to facilitate inserting the elongated rod via said incision into the surgical site;
and an inflatable balloon carried by the outer surface of the elongated rod adjacent to said distal end;
the interior of said balloon communicating with said lumen such that the portion of the elongated rod carrying said balloon may be inserted, by pressing said tapered distal end through said incision, into a surgical site, with the balloon in its deflated condition and located between the tissue layers to be separated, whereupon the balloon may be inflated by a fluid introduced into said lumen via the proximal end of the elongated rod to thereby separate said tissue layers.

17. The medical implement according to claim 10, wherein said proximal end of the elongated rod includes a valve which may be selectively opened to permit inflation of said balloon, or closed to maintain said balloon inflated.

18. The medical implement according to claim 16, wherein balloon is of triangular configuration having a center coaxial with the axis of said rod, first and second apices defining a base for engagement with one tissue layer, and a third apex engageable with a second tissue layer effective, upon the inflation of said balloon, to separate said tissue layers.

19. In the performance of an arthroscopic surgical procedure in the shoulder of a patient, the method of creating a free space between the deltoid muscle and the humerus bone at a surgical site, comprising:
inserting into said surgical site an elongated rod having a tapered proximal end carrying an inflatable balloon;
locating the balloon, while deflated, between the deltoid muscle and the humerus bone;
and inflating said balloon.

20. In the performance of an arthroscopic surgical procedure in the shoulder of a patient, the method of creating a free space between the glenoid socket and the humerus bone at a surgical site, comprising:
inserting into said surgical site an elongated rod having a tapered proximal end carrying an inflatable balloon;
locating the balloon, while deflated, between the glenoid socket and the humerus bone;
and inflating said balloon.
